(19) United States
(12) Plant Patent Application Publication
(10) Pub. No.: US 2014/0053301 P1
(43) Pub. Date: Feb. 20, 2014

(54) Apple tree named MAIA1

(50) ;

(71) Applicants: William Dodd, Newcomerstown, OH (US); David Doud, Wabash, IN (US); John Mitchell Lynd, Pataskala, OH (US); Gregory Miller, Carrollton, OH (US)

(72) Inventors: William Dodd, Newcomerstown, OH (US); David Doud, Wabash, IN (US); John Mitchell Lynd, Pataskala, OH (US); Gregory Miller, Carrollton, OH (US)

(21) Appl. No.: 13/573,033

(22) Filed: Aug. 15, 2012

(51) Int. Cl. A01H 5/00

(52) U.S. Cl. PLT/168

(57) ABSTRACT

A new and distinct variety of apple, MAIA1, is described, derived from a cross of ‘Honeycrisp’ and ‘Fuji’. This new variety is unique from parent ‘Honeycrisp’ by being harvested in mid-October, 6-8 weeks after ‘Honeycrisp’. This new variety is unique from parent ‘Fuji’ by tree form, with MAIA1 being moderate vigor, more open tree canopy and higher spur density as contrasted with the bushy, tight tree canopy and lower spur density of ‘Fuji’. MAIA1 blooms approximately one week later than parent ‘Fuji’. MAIA1 fruit is moderate size, very crisp, sweet and long keeping, both in refrigerated storage and at room temperature.

### LATIN NAME OF THE GENUS AND SPECIES OF THE PLANT CLAIMED

Malus×domestica

### VARIETY DENOMINATION

MAIA1

### BACKGROUND OF THE INVENTION

A new and distinct variety of apple was identified from a population of seedlings derived from a cross of ‘Honeycrisp’ (U.S. Plant Pat. No. 7,197) and ‘Fuji’ (not patented—original strain originating from a cross of ‘Delicious’ and ‘Ralls Janet’ made in Japan and released as a variety in 1962). This cross was made as a part of the Midwest Apple Improvement Association apple breeding project. This superior seedling tree was identified in a population of seedlings from this cross at David Doud's Countyline Orchard, Wabash, Ind.

The seedling tree was planted as a 1 year old tree at David Doud's Countyline Orchard, Wabash, Ind. in 2001 and grown among a population of several hundred siblings. Evaluations of fruit quality and tree growth parameters were begun in 2005 and this seedling was identified over several years as superior based upon tree growth habit, precocity, superior fruit quality and storability. Utilizing grafting reproduction, the new apple tree variety was asexually propagated by David Doud in 2008 at Countyline Orchard, Wabash, Ind. and has been observed to remain true to the description set forth herein.

The new variety, named MAIA1 is distinct from ‘Honeycrisp’ as MAIA1 ripens mid-October, 6-8 weeks after ‘Honeycrisp’ (Table 1). MAIA1 is distinct from ‘Fuji’ by tree growth habit, with MAIA1 being less vigorous, more open canopy, with increased number of fruit spurs per foot of 2 year wood (Table 2; FIG. 2). In addition, MAIA1 blooms approximately one week later than parent ‘Fuji’. MAIA1 fruit (FIG. 3) are medium sized, extremely crisp, sweet (Table 3), with long storability.

### BRIEF DESCRIPTION OF THE DRAWINGS

The accompanying photographs show typical specimens of the new variety as depicted in color as nearly true as is reasonably possible. MAIA1 photographs were taken by David Doud, at County Line Orchard, Wabash, Ind.; ‘Fuji’ photographs were taken by Mitch Lynd, at Lynd Fruit Farm, Pataskala, Ohio.

FIG. 1. Original seedling tree of MAIA1 showing open canopy and moderate tree vigor.

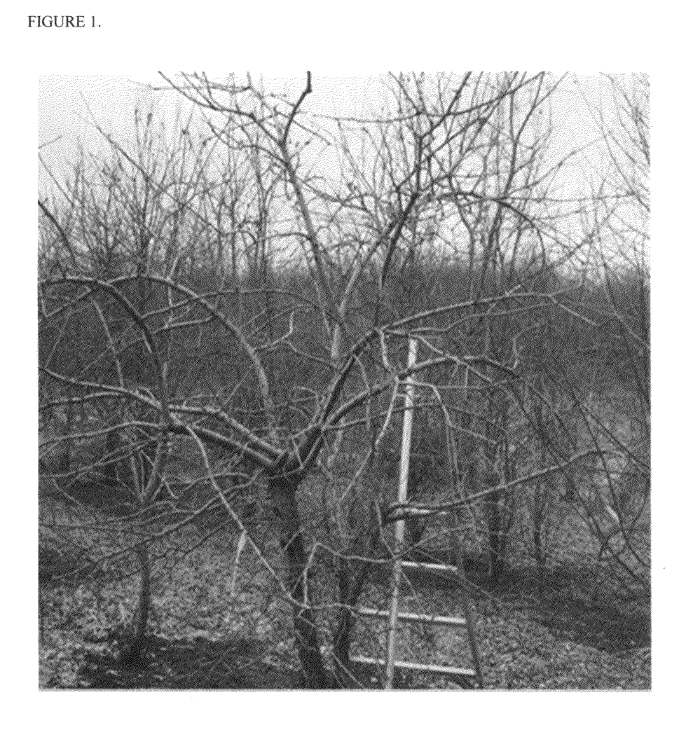

FIG. 2. One and two year old wood of original seedling tree of MAIA1, showing high spur density.

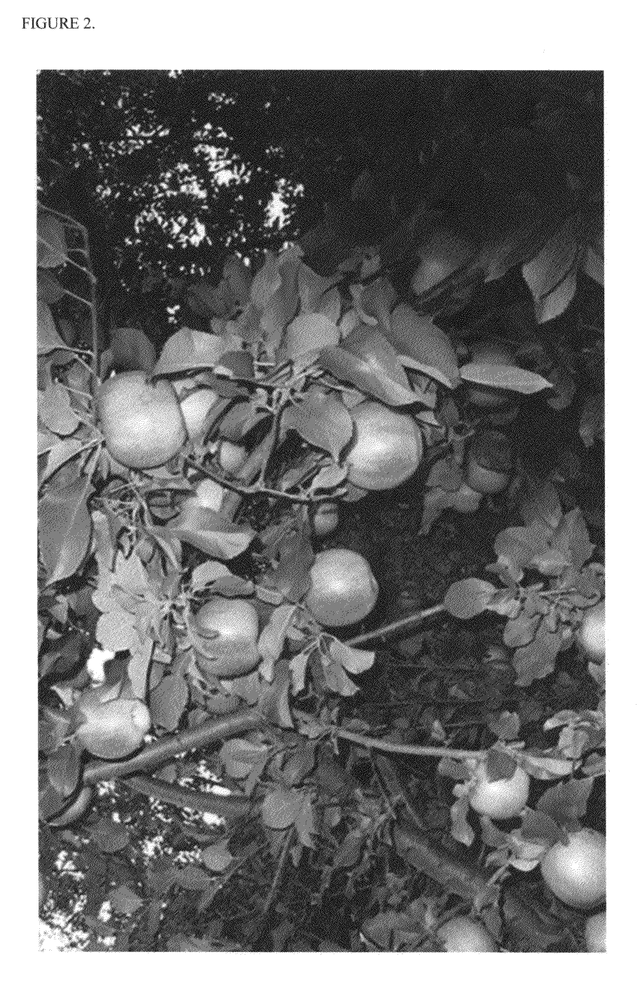

FIG. 3. Fruit of MAIA1 on the tree, October 2011.

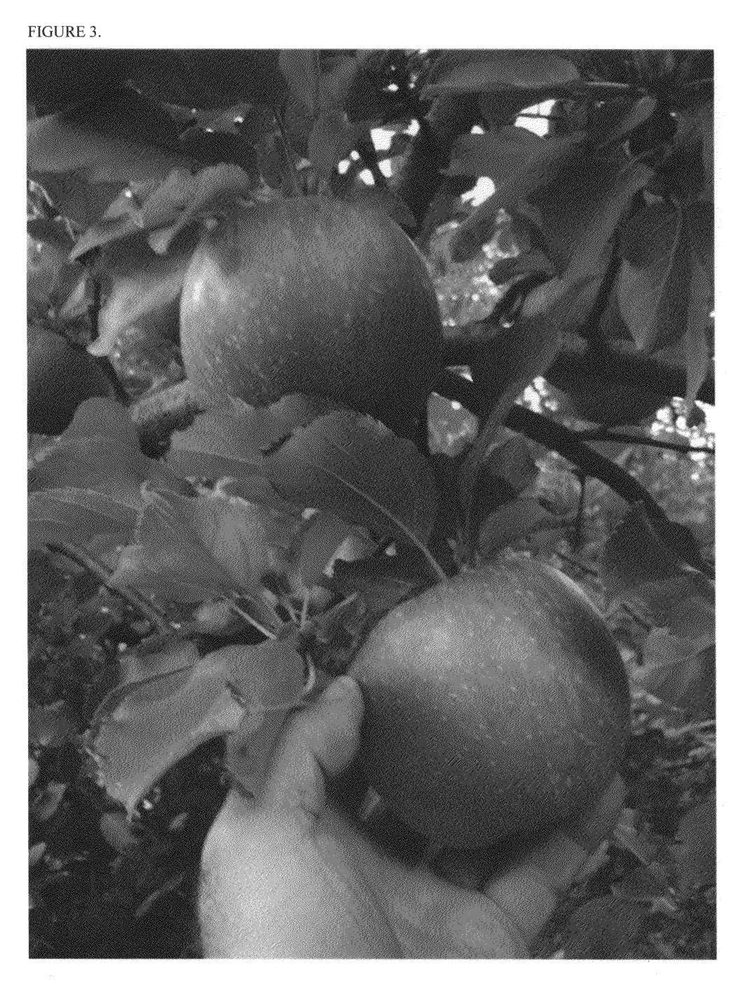

### DETAILED BOTANICAL DESCRIPTION

Color references are made to The Royal Horticultural Society Color Chart (R.H.S.) 2001 Edition.
Parentage: ‘Honeycrisp’ female parent and ‘Fuji’ male parent; controlled cross made by Greg Miller at Lynd Fruit Farm, Pataskala, Ohio in 1999.
Original seedling tree:
   Age.—12 years.
   Size.—10′ height, 10′ spread.
   Vigor.—low to medium.
   Density.—medium.
   Form.—round, spreading.
   Production.—very productive; 2.5 bushels/tree @ 300 trees/acre=750 bushels/acre.
   Growth type.—non-spur; very weak dominance.
   Bearing.—annual.
Trunk:
   Original seedling tree size.—13 cm at 15 cm height.
   Bark color.—Grey-Brown 199A.
   Lenticels.—oblong, 1.5 mm×2.5 mm.
   Lenticel color.—Grey-Brown 199B.
   Lenticel density.—6 lenticels/cm².
Branches:
   3 year old.—16-22 mm in diameter, branch angle 80-90°, Grey-Brown 199A.
   2 year old.—14-18 mm in diameter, branch angle 80°-90°, Grey-Brown 199A.
   1 year old.—9-10 mm in diameter, branch angle 80°-90°, Grey-Brown 199D.
Leaves:
   Size.—length 75 mm; width 55 mm.
   Texture.—leathery, crisp.
   Form.—ovate.
   Base.—roundly cuneate.
   Apex.—acute.
   Adaxial surface pubescence.—none.
   Abaxial pubescence.—finely pubescent.
   Adaxial surface color.—Yellow-Green 146B.
   Abaxial surface color.—Yellow-Green 146D Veination: pinnate, 8-9 major veins; mainly alternate; Yellow-Green 149D.
   Margin.—serrate.
   Petiole length.—30 mm.
   Petiole width.—3 mm Petiole abaxial color: Yellow-Green 145A to Greyed-Purple 185C toward base and abscission layer on larger leaves.
   Petiole groove.—very shallow, depth 0.1 mm Stipules: present; 5 mm length; 1 mm width; Yellow-Green 146D.
   Leaf glands.—none observed.
Leaf buds:
   Length.—length 1.5 mm.
   Width.—1.5 mm.
   Color.—Yellow-Green 145B with Greyed-Purple 185B overtones.
   Placement on branch.—alternate.
   Internode distance.—25 mm.
Spurs: (Present on 2 yr and older wood)
   Length.—varies from 10 to 350 mm.
   Proportion.—350 mm=1 200-350 mm=3 100-200 mm=3 50-100 mm=3 10-50 mm=3.
   Width.—short; 3.5 mm medium: 5 mm long: 7 mm.
Flowers at popcorn stage:
   Pedicel.—length 12-15 mm; diameter 2 mm.
   Pedicel color.—Yellow-Green 148D.
   Bud.—length 7-9 mm; width 7 mm.
   Bud color.—Red-Purple 63B, before flower petals expand and open; background of White 155C.
Flowers at full bloom:
   Bloom time.—depends on spring temperatures, but with ‘Golden Delicious’. MAIA1 blooms approximately one week later than parent ‘Fuji’.
   Bloom period.—depends on spring temperatures but first spur blossoms and then lateral blossoms on one-year wood; approximately 7 days.
   Presentation.—showy.
   Fragrance.—aromatic.
   Fertility.—fertile.
   Corolla diameter.—35-40 mm.
   Number of flowers per cluster.—4-6 with 5 typical.
Petals:
   Arrangement.—bases overlapping; 5 petals/flower; each petal 15-18 mm length; 12-14 mm width.
   Color.—upper surface (inside) White 155C; lower surface (outside) White 155C with Red-Purple 70B overtones pronounced as flower begins to open and then fading as flower completely opens.
   Shape.—broadly ovate, abruptly cuneate at junction with receptacle.
   Veins.—non-distinct.
   Margins.—somewhat ruffled with occasional notching at the apex.
   Texture.—soft.
   Receptacle.—length 8 mm; width 6 mm; color Green 143D.
   Pedicel.—length 18-22 mm; width 2 mm; color Green 143D when pubescent; Reddish tones 58A as pubescence is lost as pedicel matures.
   Sepals.—5/flower; wedge shaped; sharply pointed; length 6 mm; width 3 mm at widest point (attachment); color Greyed-Green 193B with Reddish Purple tip 58A.
   Stamens.—18-22 in number.
   Anthers.—length 2 mm; width 1.5 mm; color yellow 3D.
   Pollen.—present.
   Filaments.—length 5-12 mm, increasing in length as flower matures; width 0.5 mm; color Greyed-Green 193B.
   Pistil.—held lower than anthers in majority of blossoms.
   Ovary.—length 4 mm; width 3 mm; color Green 139B in cross section with Reddish Purple 60A layer under exterior pubescence.
   Style.—length 10 mm from fused base to stigmas; width 1.5 mm at fused base; width 0.4 mm as separated styles; pubescent at point of separation from fused base; color Green 143A.
   Stigma.—width 1 mm; color Green 143A.
Pollination requirements: Requires cross pollination from diploid varieties with overlapping bloom; will pollinate diploid varieties of overlapping bloom; both pollen and female flowers have been used successfully as parents in MAIA breeding program
Fruit:
   Maturity when described.—4 month storage.
   Date of picking.—10-30-11.
   Size.—Axial diameter 65-70 mm, Transverse diameter 75-80 mm.
   Fruit weight.—180-220 g, average 200 g.
   Form.—roundish oblate regular.
   Cavity.—acuminate medium deep; russet extending out of cavity.
   Basin.—(shape, depth, width) medium depth, medium width, symetrical obtuse regular wavy.
   Calyx.—closed reflexed.
Skin:
   Thickness.—medium.
   Tendency to crack.—little.
   Lenticels.—round shape; 0.02-0.08 mm diameter; areolar.
   Color.—Orange-Red N 34 A Stripes yes/light striping.
   Ground color.—Orange-White 159 B.
Flesh:
   Aroma.—sweet, aromatic.
   Color.—Yellow-White 158 C.
   Texture.—firm, crisp, breaking, juicy, sweet.
   Eating quality.—excellent.
Core: Medium size
   Bundle area.—250 mm.
   Bundle.—inconspicuous.
   Carpillary area.—80 mm.
   Seed cells.—walls thin, tough.
Seeds:
   Number perfect.—10.
   Number in one cell.—2.
   Length.—9 mm.
   Breadth.—5 mm.
   Color.—Greyed-Yellow 162 A; seeds often partially covered with white (presumed calcium) deposits.
Stem:
   Length.—20-30 mm.
   Width.—1.8 mm.
   Color.—Greyed-Yellow 162 A.
Use: Fresh market, dessert
Shipping quality: Good, subject to stem puncture
Keeping quality: Excellent
Tree winter hardiness: Average for an apple variety; depending on acclimation tree is hardy to −10 to −25°
Drought tolerance: Average for an apple variety. Normal requirements average ½″ of rain per week during the growing season.
Disease resistance: Susceptible to apple scab (Venturia inaequalis), powdery mildew (Podosphaera leucotricha) and other fungal diseases; slight resistance to fireblight (Erwinia amylovora); fruit susceptible to soft rot (Penicillium expansum).

TABLE 1
Harvest time of ‘Honeycrisp’ and MAIA1, 2008-2011

| | Year | Year | Year | Year |
|---|---|---|---|---|
| Variety | 2008 | 2009 | 2010 | 2011 |
| ‘Honeycrisp’ | 30-Aug | 1-Sep | 27-Aug | 27-Aug |
| MAIA1 | 25-Oct | 25-Oct | 22-Oct | 17-Oct |

TABLE 2
Fruiting Spur Density on 2-year old wood
of ‘Fuji’ and MAIA1, 2011

| | number of spurs/ft of 2- |
|---|---|
| Variety | year-old wood |
| ‘Fuji’ | 1-5 |
| MAIA1 | 8-15 |

TABLE 3
Fruit quality data-Fabulous Food Show, November 2010
FRUIT QUALITY

| | | ‘Honeycrisp’ | ‘Fuji’ | MAIA1 |
|---|---|---|---|---|
| Parameter | | | | |
| weight (g) | | 226 | 200.2 | 215 |
| diameter (mm) | | 83 | 78.2 | 77.8 |
| length (mm) | | 68.5 | 68.5 | 67.9 |
| shape index (1/d) | | 0.8253 | 0.876 | 0.8728 |
| cover color | | | | |
| | % | 75.3 | 81.7 | 90.3 |
| | L | 43.2 | 44.9 | 42.8 |
| | C | 37.8 | 36.2 | 37.4 |
| | h | 33.8 | 32.9 | 30.6 |
| background color | | | | |
| | % | 24.7 | 18.3 | 9.7 |
| | L | 74.9 | 68.7 | 70.9 |
| | C | 42.3 | 42.4 | 38.5 |
| | h | 101.2 | 93.7 | 99.5 |
| Soluble Solids (%) | | 14.3 | 17.2 | 15.6 |
| Titratable Acidity (%) | | 4.7 | 4.3 | 5.1 |
| Firmness (kg/cm2) | | 7.2 | 7.5 | 7.9 |

What is claimed is:

1. A new and distinct apple tree variety named ‘MAIA1’, as illustrated and described herein.
